(19) United States
(12) Patent Application Publication
(10) Pub. No.: US 2025/0006367 A1
(43) Pub. Date: Jan. 2, 2025

(54) SYNTHETIC DIGITAL TWIN FOR A PATIENT

(71) Applicant: INTERNATIONAL BUSINESS MACHINES CORPORATION, ARMONK, NY (US)

(72) Inventors: Chen Wang, Chappaqua, NY (US); Bo Wen, New York, NY (US); Erhan Bilal, Westport, CT (US)

(21) Appl. No.: 18/345,537

(22) Filed: Jun. 30, 2023

(51) Int. Cl. G16H 50/20; G16H 10/60; G16H 50/70

(52) U.S. Cl. CPC G16H 50/20; G16H 10/60; G16H 50/70

(57) ABSTRACT

A method, computer program product, and computer system for generating synthetic time-series data for a specific disease. One or more processors of a computer system provide a generative adversarial network (GAN), train the GAN to generate time series data using episodic measurement results as metadata for a patient cohort with a specific disease; receive input metadata associated with an episodic measurement for a patient in the patient cohort with the specific disease by the trained GAN, and generate synthetic time series data that simulates the patient in the patient cohort with the specific disease.

### BACKGROUND

The present invention relates to creating synthetic digital twins, and more specifically, to creating a synthetic digital twin for a patient in a healthcare setting, using a generative adversarial network (GAN).

### SUMMARY

Embodiments of the present invention provide a method, a computer program product, and a computer system, for generating synthetic time-series data for a specific disease. One or more processors of a computer system provide a generative adversarial network (GAN), train the GAN to generate time series data using episodic measurement results as metadata for a patient cohort with a specific disease, receive input metadata associated with an episodic measurement for a patient in the patient cohort with the specific disease by the trained GAN, and generate synthetic time series data that simulates the patient in the patient cohort with the specific disease.

### BRIEF DESCRIPTION OF THE DRAWINGS

FIG. 1 depicts a computing environment which contains an example of an environment for the execution of at least some of the computer code involved in performing the inventive methods, in accordance with embodiments of the present invention.

FIG. 2 depicts a block diagram of modules included in computer code included in the system of FIG. 1, in accordance with embodiments of the present invention.

FIG. 3 depicts a flow chart of an embodiment of a method for training a GAN, in accordance with embodiments of the present invention.

FIG. 4 depicts a flow chart of an embodiment of a method for generating synthetic time-series data for a specific disease, in accordance with embodiments of the present invention.

FIG. 5 depicts a flow chart of an embodiment of a method for discriminating, in accordance with embodiments of the present invention.

FIG. 6 depicts a process flow architecture for generating synthetic time-series data for a specific disease, in accordance with embodiments of the present invention.

FIG. 7 depicts implementation steps to adapt a DoppelGANger architecture for generating synthetic time-series data for a specific disease, in accordance with embodiments of the present invention.

### DETAILED DESCRIPTION

### Overview

Digital twin technology is a relatively new concept that has the potential to revolutionize healthcare. It involves the creation of a virtual replica or “twin” of a patient, which can then be used to simulate and analyze the patient's behavior. This virtual twin can provide valuable insights and enable healthcare professionals to make more informed decisions about the patient's care. For example, a digital twin of a patient could be used to predict the likelihood of certain health outcomes or to optimize treatment plans based on individualized data. Overall, digital twin technology has the potential to improve patient care, reduce costs, and increase the efficiency of the healthcare system by enabling healthcare professionals to make more informed decisions based on data-driven simulations.

DoppelGANger is a categorical generative adversarial network (GAN). It is the only known method that supports synthesizing high-fidelity time series datasets using metadata labels (categorical information). It has been proven helpful in stock market analysis and cloud/data center performance analysis.

Today, most digital twin applications in healthcare are still as simple as merely a new form of the electronic health record, i.e., adding wearable/IoT data on top of the traditional data collected in clinics. The potential of the new data streams is not being fully leveraged.

Normally, these digital twin datasets contain both episodic measurement results (discrete data) from clinic visits and regular health checkups, such as blood test results, MRI scans, psychiatric evaluations, mobility tests, etc., and at home measurements from wearable and IoT devices, which normally in the form of time-series data. Because of the large difference in the time scale (months vs. seconds/milliseconds), modeling the relationship between the episodic measurements and the continuous time-series dataset has been challenging for the field.

The DoppelGANger technology provides a solution to establish the connection between the episodic and time-series data, and we can use it to simulate the evolvement of the digital twin of patients. This provides a tool to help physicians to understand the patient's potential disease progression trajectory. Using the DoppelGANger method, the present invention contemplates treating the episodic measurement results as metadata in order to train the GAN to generate the corresponding time series data.

After training such a GAN on a patient cohort with a specific disease, the present invention contemplates generating synthetic data that resembles real patient health data with that disease. This synthetic data can then be used to allow doctors to explore potential disease trajectories by changing input metadata. For example, methods described herein can determine what will happen to the heart rate and mobility time series for a patient if that patient gains another 5 lbs.

Diseases as contemplated herein may mean any illness, sickness, virus, health disorder, or the like. Diseases contemplated herein may be any type of medical diagnosis, and may include physiological ailments, mental illnesses, or the like. Diseases as contemplated herein may be any type of health condition in which a patient cohort group can be categorized, for example.

Using a well-trained DoppelGANger model in accordance to embodiments herein may provide for the following benefits: 1) Fidelity: Ensuring the synthetic data is realistic and solid enough to be helpful in the use cases mentioned below; 2) Diversity: Ensuring that the synthetic data is diverse enough to represent the range of variation in the real data; 3) Categorizable: Ensuring that the synthetic data is generated in a way that preserves detailed statistical properties of the real data (e.g., the distribution of values, correlations between different variables) within specific categorical labels, such as healthy or not; 4) Privacy: Ensuring that the synthetic data does not accidentally reveal sensitive information about real patients.

Prior to embodiments described herein, the DoppelGANger method has only been used in financial applications and cloud monitoring applications. As contemplated by the present invention, using the DoppelGANger method in the healthcare setting using the present methods and systems described will provide for the above-outlined benefits and advantages.

In typical healthcare data analysis and model building practices, healthcare providers use time-series data to predict the episodic measurements. In contrast, the present invention contemplates doing the reverse: i.e., using episodic data to generate/predict time-series data using a GAN. While the present invention contemplates specifically using DoppelGANger as a categorical GAN, it should be understood that other GAN methods are contemplated.

### Computing Environment

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

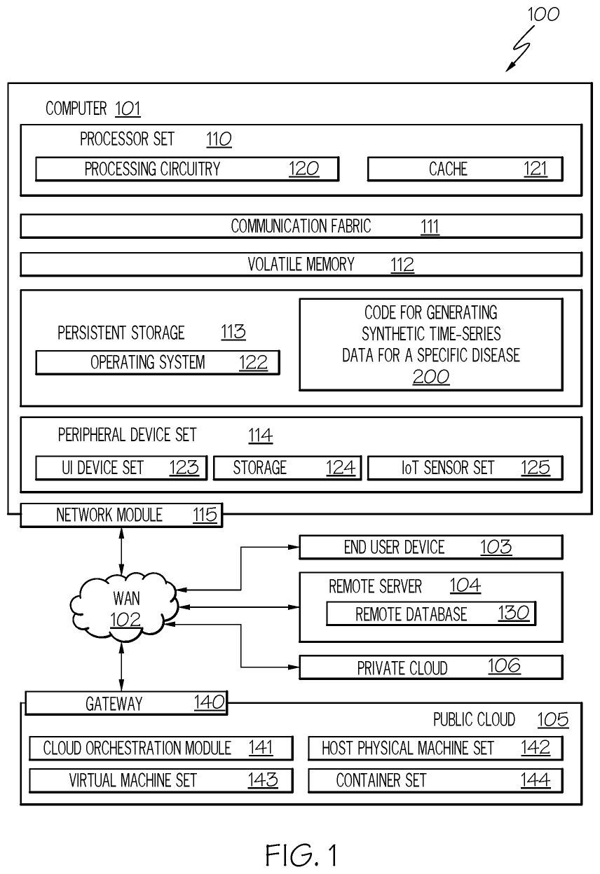

FIG. 1 depicts a computing environment 100 which contains an example of an environment for the execution of at least some of the computer code involved in performing the inventive methods, such as code 200 for generating synthetic time-series data for a specific disease. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 012 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

### Process and System for Generating Synthetic Data for a Disease

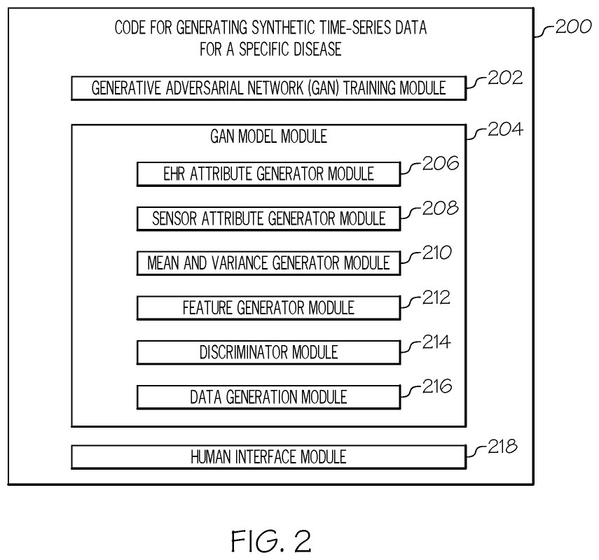

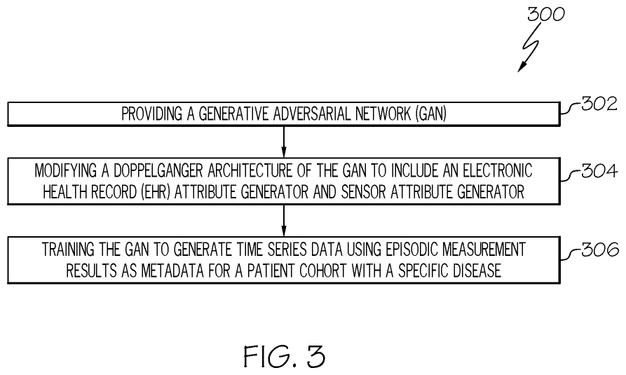

FIG. 2 is a block diagram of modules included in code for generating synthetic time-series data for a specific disease 200, included in the system of FIG. 1, in accordance with embodiments of the present invention. The code for generating synthetic time-series data for a specific disease 200 includes a generative adversarial network (GAN) training module 202, a GAN model module 204, and a human interface module 218. The GAN model module 204 further includes several sub-modules including an (Electronic Health Record) EHR attribute generator module 206, a sensor attribute generator module 208, a mean and variance generator module 210, a feature generator module 212, a discriminator module 214, and a data generation module 216. The number of modules can vary, and some modules may be combined with other modules or separated into two or more modules in various combinations. The functionality of the modules included in code for generating synthetic time-series data for a specific disease 200 is discussed in detail with respect to the methodology shown in FIGS. 3-5, which is presented below.

FIG. 3 depicts a flow chart of an embodiment of a method 300 for training a GAN, in accordance with embodiments of the present invention. The method 300 includes a first step 302 of providing a generative adversarial network (GAN). The GAN may be a type of machine learning framework that includes two neural networks which contest with each other, where gain for one neural network is a loss for another. Given a training set, the GAN may be configured to generate new data with the same statistics as the training set. The GAN may be implemented in a DoppelGANger architecture. In particular, a DoppelGANger architecture may be a categorical GAN which synthesizes high-fidelity time series datasets using metadata labels.

As contemplated herein, the DoppelGANger architecture may be configured to separately generate normalized time series data, and statistic characterizations (e.g., mean and/or variance) conditioned on sample attributes. The DoppelGANger architecture may leverage the mean and/or variance generator to ensure the variability of generated data. To strengthen temporal correlations in time series, the DoppelGANger architecture contemplated herein outputs batched samples rather than singletons by connecting a Multi-layer perception (i.e., MLP) network or other common neural network with a recurrent neural network (RNN). Batch generation may be particularly useful in healthcare use cases, because disease progression can last a few months even years, generating a limited length of timeseries data that would not have enough data to provide a practical useful patient case.

A DoppelGANger architecture as contemplated herein includes a feature generator, a mean and variance generator, and a discriminator. The method 300 includes a step 304 of modifying a typical DoppelGANger architecture to include an Electronic Health Record (EHR) attribute generator (i.e., included in the EHR attribute generator module 206) and a sensor attribute generator (i.e., included in the sensor attribute generator module 208), as well as providing an auxiliary discriminator (part of the discriminator module 214) for determining whether the meta-attributes from the EHR attribute generator, the sensor attribute generator and the mean and/or variance attribute generator are real or fake. The modified DoppelGANger architecture may further include the mean and variance generator module 210, the feature generator module 212, and the discriminator module 214 may include a conventional discriminator and the auxiliary discriminator.

The method 300 then includes a final step 306 of training the GAN to generate time series data using episodic measurement results as metadata for a patient cohort with a specific disease. The steps 302, 304, 306 of the method 300 of providing and training the modified GAN may be performed by the GAN training module 202, in combination with the GAN model module 204.

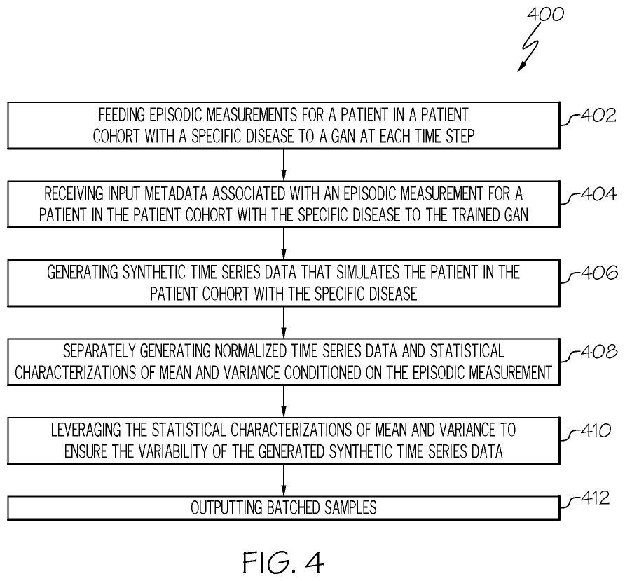

FIG. 4 depicts a flow chart of an embodiment of a method 400 for generating synthetic time-series data for a specific disease, in accordance with embodiments of the present invention. As contemplated, steps of the method 400 may be performed by the GAN model module 204, and particularly the data generation module 216, although the various other modules 206, 208, 210, 212, 214 may also be utilized in generating the data.

The method 400 includes a first step 402 of feeding episodic measurements (i.e. attributes) for a patient in a patient cohort with a specific disease to a GAN, such as the GAN trained in the method 300. This feeding episodic measurements may occur at each time step output or generated by the GAN. The feeding measurements to the GAN may be conducted by the human interface module 218, in some embodiments. In other embodiments, measurements or attributes of a patient may be provided to the GAN in an automated fashion without human interaction. Whatever the data input form, the method 400 may include a step 404 of receiving, by a computer system operating the GAN, input metadata associated with an episodic measurement for a patient in the patient cohort with the specific disease.

The method 400 then includes a step 406 of generating synthetic time series data that simulates the patient in the patient cohort with the specific disease. Step 406 may be conducted by the data generation module 216 in combination with the other modules 206, 208, 210, 212, 214 operating the GAN model module 204. Again, during this generation step, the episodic measurements (i.e., patient attributes) may be fed at each time step generated. The method 400 may further include a step 408 of separately generating normalized time series data and statistical characterizations of mean and variance conditioned on the episodic measurement(s) (i.e. patient attributes). A step 410 includes leveraging the statistical characterizations of mean and variance to ensure the variability of the generated synthetic time series data. The method 400 finally includes a step 412 of outputting batched samples (i.e. rather than singletons). The batched samples may be configured to strengthen temporal correlates in time series. The outputting batched samples may be performed by connecting the MLP with the RNNs.

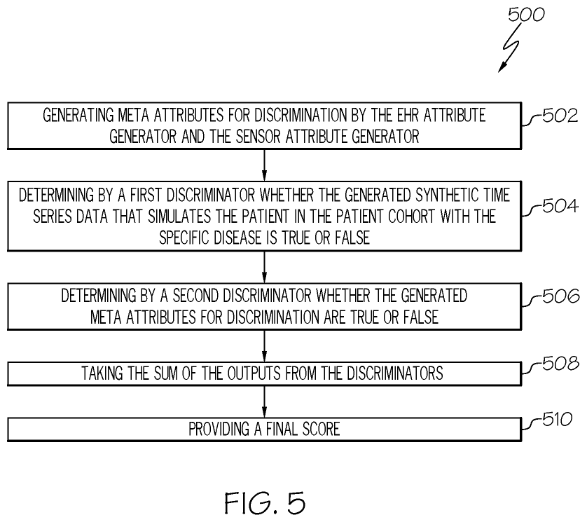

FIG. 5 depicts a flow chart of an embodiment of a method 500 for discriminating, in accordance with embodiments of the present invention. The method 500 may be largely conducted by the discriminator module 214 of the GAN model module 204. The method 500 includes a step 502 of generating meta attributes for discrimination by the EHR attribute generator (from the EHR attribute generator module 206) and the sensor attribute generator (from the sensor attribute generator module 208). With the outputs from these modules 206, 208, and further from the mean and variance generator module 210 and the feature generator module 212, the method 500 includes a step 504 of determining by a first discriminator of the discriminator module 214, whether the generated synthetic time series data that simulates the patient in the patient cohort with the specific disease is true or false. The method 500 includes a step 506 of determining, by a second discriminator of the discriminator module 214, whether the generated meta attributes for discrimination are true or false. The method 500 includes a step 508 of taking the sum of the outputs from the discriminators by the discriminator module 214 and a final step 510 of providing a final score.

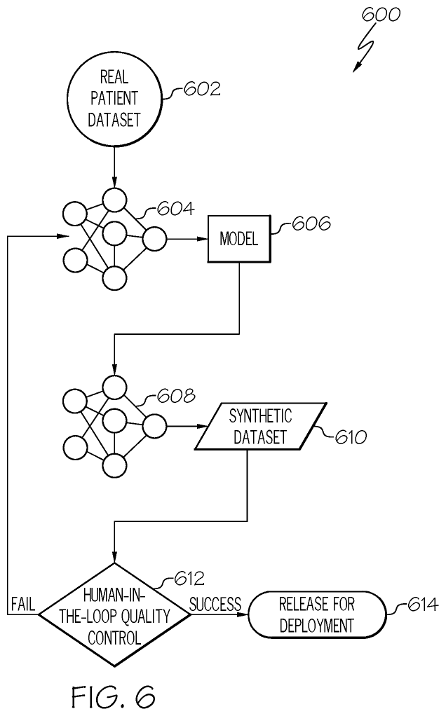

FIG. 6 depicts a process flow architecture 600 for generating synthetic time-series data for a specific disease, in accordance with embodiments of the present invention. The architecture 600 includes a first process of inputting a real patient dataset 602 to a GAN training system 604, such as the GAN training module 202. The real patient dataset 602 may include training data for model training of the GAN training system 604. The architecture 600 then includes creating a model 606 by the GAN training system 604 from the real patient dataset 602. The model 606 may be a modified DoppelGANger model, as described herein. Once a model is generated, time series data generation may occur using the model 606 at the data generation process 608, whereby a synthetic time series dataset 610 may be produced by the model 606. The data generation process 608 may occur based on an input of one or more real attributes or patient measurements (i.e. episodic measurements), whereby a synthetic dataset is generated based on these attributes.

At a step 612, a human-in-the-loop quality control process 612 occurs whereby a human or user of the system may be provided with an opportunity to determine whether the resulting synthetic dataset 610 is acceptable or unacceptable. If the human or user determines that the synthetic dataset 610 is unrealistic, the failure will be provided back to the GAN training system 604 for updating the model 606 to account for the failure. If the human or user determines that the synthetic dataset 610 is a success, then the model 606 may be released for further deployment at a process 614. The architecture 600 thereby provides for the creation and generation of the model 606 in accordance with the principles described herein, and the possible iterative updating of the model until a quality control system determines that the model is acceptable.

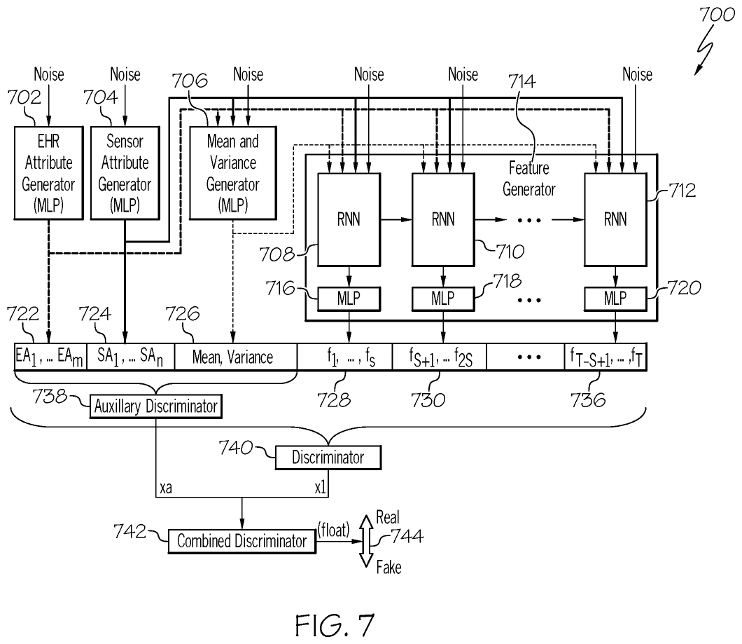

FIG. 7 depicts implementation steps to adapt a DoppelGANger architecture 700 for generating synthetic time-series data for a specific disease, in accordance with embodiments of the present invention. The DoppelGANger architecture 700 includes an EHR attribute generator 702 for generating HER attributes 722, a sensor attribute generator 704 for generating sensor attributes 724, and a mean and variance generator 706 for generating mean and variance 726. Each of the EHR attribute generator 702, the sensor attribute generator 704 and the mean and variance generator 706 may include a Multi-layer perception (i.e., MLP) network.

The EHR attribute generator 702 and the sensor attribute generator 704 may be decoupled from the feature generator 714. To learn better correlations between time series and their attributes, the typical DoppelGANger architecture has been modified to include the EHR attribute generator 702 and the sensor attribute generator 704 to better accommodate the digital twin of the patient by decoupling the generation of attributes from the time series and feeding attributes to the time series generator at each time step. This is advantageous over conventional approaches, whereby attributes and features are generated jointly. This conditional generation architecture also provides improved flexibility to change the attribute distribution without sacrificing fidelity. It also enables the hiding of real attribute distribution when there is a privacy concern in the healthcare environment.

Similar to the EHR attribute generator 702 and the sensor attribute generator 704, the mean and variance generator 706 is also decoupled from the feature generator 714. It has been observed that traditional GANs trained on datasets with a highly variable dynamic range across samples tend to exhibit severe mode collapse where the generator always outputs very similar samples. The present GAN implementations typically relate systems that use a narrow class of signals (e.g., images); in contrast, networking time series data as contemplated herein exhibits much more variability across each sample's max/min limits. To address this, the present invention contemplates using a DoppelGANger architecture to separately generate normalized time series data and statistical characterizations (e.g., mean and variance) conditioned on the sample attributes. With the variabilities in the samples of patents' data, the present invention contemplates leveraging the mean and variance generator 706 to ensure the variability of the generated data.

Batched generation occurs in the feature generator 714 for generating features 728, 730, 736, which includes a plurality of RNNs 708, 710, 712. To strengthen temporal correlations in time series, the DoppelGANger outputs batched samples rather than singletons by connecting the Multi-layer perception (MLP) networks 716, 718, 720, with the RNNs 708, 710, 712, respectively. Batch generation may be particularly useful in healthcare use cases because disease progression can last months or years. Generating a limited length of timeseries may not be able to synthesize enough data to represent a practical useful patient case. Embodiments herein contemplate reusing and leveraging the feature generator 714 of the DoppleGANger's architecture 700.

The DoppleGANger's architecture 700 further includes an auxiliary discriminator 738. The auxiliary discriminator 738 may be a first discriminator that is trained to determine whether the “meta attributes” 722, 724, 726 are true or fake. These attributes are generated by the EHR attribute generator, the Sensor Attribute generator, and the Mean/Variance attribute generator.

The DoppleGANger's architecture 700 further includes a second discriminator 740 that is trained to determine whether the generated timeseries output of the feature generator 714 is true or fake.

The DoppleGANger's architecture 700 further includes a combined discriminator 742. The outputs of the first and second discriminators 738, 740 may be provided to a combined discriminator 742 so that the combined discriminator 742 can that take the sum of the outputs from the first and second discriminators 738, 740, to provide a final discrimination score 744 as a float number.

Various use case examples are contemplated using the above-described embodiments. For example, the computing environment 100, code 200, methods 300, 400, 500, process flow 600 and architecture 700 may be used in various settings. One exemplary setting may be virtual clinical trials. In such an embodiment, it may be expensive and sometimes difficult to recruit enough patients to fill the entire parameter space. For example, in a comorbidity study, demographic parameters or attributes such as age, gender, body weight, and height might be easy to obtain by recruiting enough people to have a uniform and balanced distribution. However, it might be difficult to find enough patients with both a specific cardiac condition and kidney disease that covers all the demographic dimensions. With a well-trained GAN model as contemplated by embodiments herein on such a patient cohort, researchers can explore the parameters space with the synthetic dataset. For example, embodiments of the present invention may train a model, such as the model 606, and input the attributes of an imaginary female patient of this specific patient cohort (i.e. having a specific cardiac condition and kidney disease), including age, weight, height attributes (e.g., age 71, 120 lbs., and 5′8″ height). A synthetic data set, such as the synthetic data set 610, in order to determine what an exemplary her heart rate data over a certain period would likely be.

Embodiments disclosed herein may be used for generating synthetic datasets to be used as part of a product demonstration and to remove the risk of leaking patient identification and other legal liabilities—as synthetic data will not include privacy issues. Still further, embodiments disclosed herein may be used for employee training. Embodiments described may be able to generate unlimited sets of different datasets with the same statistical features to be used in employee training programs for people to learn how to conduct analysis on real time-series data.

In another exemplary embodiment, polysomnography studies (PSG) record EEG, 02, EKG, and other signals during sleep. It is an expensive and relatively uncomfortable procedure that involves a patient sleeping at a specialized clinic while connected to numerous sensors. In PSG studies, a sleep technician labels the data in 30-second batches for apnea events or other sleep disorders. Other labels or attributes (i.e., metadata) can be collected from the patient's clinical data: age, weight, BMI, etc. Using the present computing environment 100, code 200, methods 300, 400, 500, process flow 600 and/or architecture 700 described herein, it is possible to cheaply simulate realistic PSG data conditioned on clinical labels. For example, once a GAN model has been trained in accordance with the embodiments described herein, it is possible to input specific attributes of a patient, and then alter the attributes of the patient in order to simulate new synthetic data. For example, one may answer the question of how the sleep breathing patterns may change in a patient suffering from sleep apnea if the patient lost 20 lbs (i.e., if a weight attribute input into the model was changed by 20 pounds and a synthetic dataset was generated.

In still further embodiments contemplated herein, recent studies showed that “brain waves” (EEG) can be interpreted and translated into specific actions or words. When applying the present computing environment 100, code 200, methods 300, 400, 500, process flow 600 and/or architecture 700 described hereinabove, to an EEG generating model, it may be possible to use speech to label brain waves by recording a patient's speech and EEG signals concurrently. Because it is known that speech can be used to diagnose mental health disease, the ability to simulate EEG data from speech could help objectively diagnose certain mental health diseases and understand them better.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
providing, by one or more processors of a computer system, a generative adversarial network (GAN);
training, by the one or more processors of the computer system, the GAN to generate time series data using episodic measurement results as metadata for a patient cohort with a specific disease;
receiving, by the trained GAN and the one or more processors of the computer system, input metadata associated with an episodic measurement for a patient in the patient cohort with the specific disease to the trained GAN; and
generating, by the trained GAN and the one or more processors of the computer system, synthetic time series data that simulates the patient in the patient cohort with the specific disease.

2. The computer-implemented method of claim 1, wherein the trained GAN is implemented in a DoppelGANger architecture.

3. The computer-implemented method of claim 2, further comprising:
modifying the DoppelGANger architecture to include an Electronic Health Record (EHR) attribute generator and sensor attribute generator, the EHR attribute generator and the sensor attribute generator each configured to generate meta attributes for discrimination.

4. The computer-implemented method of claim 3, further comprising:
determining, by a first discriminator of the trained GAN, whether the generated synthetic time series data that simulates the patient in the patient cohort with the specific disease is true or false.
determining, by an auxiliary discriminator of the trained GAN, whether the generated meta attributes for discrimination are true or false.

5. The computer-implemented method of claim 1, further comprising:
feeding the episodic measurement for a patient in the patient cohort with the specific disease to the GAN at each time step.

6. The computer-implemented method of claim 1, wherein the generating, by the trained GAN and the one or more processors of the computer system, synthetic time series data that simulates the patient in the patient cohort with the specific disease further comprises:
separately generating, by the trained GAN and the one or more processors of the computer system, normalized time series data and statistical characterizations of mean and variance conditioned on the episodic measurement; and
leveraging, by the one or more processors of the computer system, the statistical characterizations of mean and variance to ensure the variability of the generated synthetic time series data.

7. The computer-implemented method of claim 1, further comprising:
outputting, by the trained GAN and the one or more processors of the computer system, batched samples of the synthetic time series data by connecting at least one multilayer perceptron (MLP) artificial neural network with at least one recurrent neural network.

8. A computer program product for generating synthetic time-series data for a specific disease, the computer program product comprising a computer readable hardware storage medium having program instructions embodied therewith, the program instructions readable by one or more processors of a computer system to cause the one or more processors to:
providing, by the one or more processors of the computer system, a generative adversarial network (GAN);
training, by the one or more processors of the computer system, the GAN to generate time series data using episodic measurement results as metadata for a patient cohort with a specific disease;
receiving, by the trained GAN and the one or more processors of the computer system, input metadata associated with an episodic measurement for a patient in the patient cohort with the specific disease to the trained GAN; and
generating, by the trained GAN and the one or more processors of the computer system, synthetic time series data that simulates the patient in the patient cohort with the specific disease.

9. The computer program product of claim 8, wherein the trained GAN is a DoppelGANger implementation.

10. The computer program product of claim 9, further comprising:
modifying the DoppelGANger architecture to include an Electronic Health Record (EHR) attribute generator and sensor attribute generator, the EHR attribute generator and the sensor attribute generator each configured to generate meta attributes for discrimination.

11. The computer program product of claim 10, further comprising:
determining, by a first discriminator of the trained GAN, whether the generated synthetic time series data that simulates the patient in the patient cohort with the specific disease is true or false.
determining, by an auxiliary discriminator of the trained GAN, whether the generated meta attributes for discrimination are true or false.

12. The computer program product of claim 8, further comprising:
feeding the episodic measurement for a patient in the patient cohort with the specific disease to the GAN at each time step.

13. The computer program product of claim 8, further comprising:
separately generating, by the trained GAN and the one or more processors of the computer system, normalized time series data and statistical characterizations of mean and variance conditioned on the episodic measurement; and
leveraging, by the one or more processors of the computer system, the statistical characterizations of mean and variance to ensure the variability of the generated synthetic time series data.

14. The computer program product of claim 8, further comprising:
outputting, by the trained GAN and the one or more processors of the computer system, batched samples of the synthetic time series data by connecting at least one common neural network with at least one recurrent neural network.

15. A computer system comprising:
one or more computer processors;
one or more computer readable storage media; and
computer readable code stored collectively in the one or more computer readable storage media, with the computer readable code including data and instructions to cause the one or more computer processors to perform at least the following operations:
providing, by the one or more processors of the computer system, a generative adversarial network (GAN);
training, by the one or more processors of the computer system, the GAN to generate time series data using episodic measurement results as metadata for a patient cohort with a specific disease;
receiving, by the trained GAN and the one or more processors of the computer system, input metadata associated with an episodic measurement for a patient in the patient cohort with the specific disease to the trained GAN; and
generating, by the trained GAN and the one or more processors of the computer system, synthetic time series data that simulates the patient in the patient cohort with the specific disease.

16. The computer system of claim 15, wherein the trained GAN is a DoppelGANger implementation.

17. The computer system of claim 16, further comprising:
modifying the DoppelGANger architecture to include an Electronic Health Record (EHR) attribute generator and sensor attribute generator, the EHR attribute generator and the sensor attribute generator each configured to generate meta attributes for discrimination.

18. The computer system of claim 17, further comprising:
determining, by a first discriminator of the trained GAN, whether the generated synthetic time series data that simulates the patient in the patient cohort with the specific disease is true or false.
determining, by an auxiliary discriminator of the trained GAN, whether the generated meta attributes for discrimination are true or false.

19. The computer system of claim 15, further comprising:
feeding the episodic measurement for a patient in the patient cohort with the specific disease to the GAN at each time step.

20. The computer system of claim 15, further comprising:
separately generating, by the trained GAN and the one or more processors of the computer system, normalized time series data and statistical characterizations of mean and variance conditioned on the episodic measurement; and
leveraging, by the one or more processors of the computer system, the statistical characterizations of mean and variance to ensure the variability of the generated synthetic time series data.
